(19) United States
(12) Patent Application Publication
(10) Pub. No.: US 2005/0010304 A1
(43) Pub. Date: Jan. 13, 2005

(54) Device and method for reconstruction of osseous skeletal defects

(76) Inventor: Amir Ali Jamali, Sacramento, CA (US)

Correspondence Address: Michael A. Oblon;SHAW PITTMAN LLP, 1650 Tysons Boulevard, McLean, VA, 22102, US

(21) Appl. No.: 10/867,748

(22) Filed: Jun. 16, 2004

Related U.S. Application Data
(60) Provisional application No. 60/478,465, filed on Jun. 16, 2003.

(51) Int. Cl. A61F 2/28; A61C 8/00

(52) U.S. Cl. 623/23.46; 623/17.11; 623/23.63; 623/20.16; 623/23.28; 433/201.1

(57) ABSTRACT

A device for the reconstruction of skeletal defects with a flexible member, which is preferably resorbable, attached to a rigid structural prosthesis such as a dental implant, an orthopedic prosthetic implant, or an artificial disc implant. The cavitary space surrounded by the flexible member is filled with osteoconductive and/or inductive materials which eventually matures into bone. The prosthesis is supported by the bed of graft material surrounding it and is gradually unloaded as the bed matures into solid bone. The fixation of the prosthesis into native bone depends on the specific implant and the anatomic area of its use. The flexible member is secured to the margins of the prosthesis using rails, runners, sutures, or other attachment devices that prevent the escape of the bone graft and maintain an initial column of support for the implant.

This application claims the benefit of U.S. Provisional Application No. 60/478,465, filed Jun. 16, 2003, which is herein incorporated by reference in its entirety.

### BACKGROUND

1. Field of the Invention

The present invention relates generally to implantable medical devices for the treatment of osseous skeletal defects, and methods for their use.

2. Background of the Invention

In the past, skeletal defects have required amputation due to the associated “flail extremity” which prohibited weightbearing due to skeletal insufficiency and lack of effective muscle power. Early in the twentieth century, Lexer popularized the transplantation of large human joint (allografts) for such problems. However, these have been associated with high rates of infections, non-unions, accelerated arthritis, and mechanical complications. With the advent of hip prosthetics as developed by Austin Moore's proximal femoral prosthesis in the 1940's and John Chamley's low friction arthroplasty (total hip arthroplasty) in the 1960's and early 1970's, some of these problems were addressed in the hip, eliminating the problem of allograft joint degeneration.

The total hip arthroplasty was later combined with allografts, forming an allograft prosthetic composite (APC), taking advantage of the healing potential between the allograft and the residual host bone as well as the relatively painfree articulation of the total joint replacement. Concurrently, segmental prostheses or “tumor prostheses” were developed. The APC and segmental prosthesis were particularly needed in the era of “limb-preservation surgery”. This concept became possible with the development of chemotherapy agents that improved survival within the field of orthopedic oncology.

These allograft prosthetic composites (APC) were associated with high risks of infection and other complications. Massive osteoarticular allografts and APC's have a tremendous disadvantage due to some residual antigenicity and the slow incorporation of the allograft bone by host bone. The process termed “creeping substitution”, whereby the allograft bone is replaced by host bone in an appositional fashion, leads to overall weakening of the graft. Large allografts have been shown to be an “admixture of necrotic and viable bone”. This is in contrast to cancellous bone which based on its three dimensional porous architecture, facilitates bone ingrowth and increased mechanical strength after implantation.

Segmental prostheses are able to span the area of bone loss and are stabilized to the residual host bone. These prostheses, however, have several problems, including their large size, the high torques at the host-prosthesis interface, and risks of dislocation due to inadequate soft tissue attachments to the metal prosthesis. These issues are commonly found in the area of the knee and hip but also apply to the shoulder, elbow, ankle, and wrist. The search is ongoing for the ideal way to address a large segmental loss of bone adjacent to a large joint.

In some cases, due to bone loss resulting from infection or debris-mediated bone digestion, termed “osteolysis”, the residual bone allows a contained defect with thin but relatively preserved walls. In such cases, a technique known as impaction grafting has been developed and used since the late 1970's. The osseous defect is serially filled with layers of cancellous bone graft, which interlock due to the force of impaction. Into this newly formed cavity, a cemented prosthesis can be inserted. As the cancellous bone graft incorporates, it restores the patient's bone stock and provides an ongoing stable bed for the cemented implant.

The common complications with the technique relate to the loss of fixation due to fracture of the host bone or lack of containment and interlock of the cancellous bed. In some cases where the host bone has a segmental defect, it can be bridged with an allograft strut or some other containment device. Alternatively, metal mesh has been used to contain the allograft. However, use of such mesh is ineffective in the event of complete deficiency of the native cortical shell due to the lack of containment of the bone graft at the end of the construct, i.e., at the hip joint in the case of a proximal femoral deficiency.

Vertebral Reconstruction

Disease of the intervertebral discs of the spine can be manifested as neck or back pain with degeneration of the central nucleus pulposis of the disc. The surrounding annulus fibrosis can tear allowing extrusion of the nucleus pulposis. The disc herniation can generate a profound inflammatory response, leading to neck or back pain as well as irritation of the spinal cord or roots. Part of the natural history of disc degeneration is gradual collapse of the disc as well as adjacent endplate degeneration, osteophyte formation, and ultimately spinal stenosis. As part of the surgical treatment of such disorders, spinal arthrodesis had enjoyed popularity in past decades. This technique suffers from disadvantages related to bony overgrowth, adjacent disc degeneration, and loss of flexibility.

Artificial discs have been developed and have suffered from suboptimal durability and only short-term follow-up in several reports. Ongoing improvements are being made. In some cases, the resection of a segment of the spine spans longer than one disc. The available options are a shortening of the spine which may lead to abnormal tension on the adjacent nerve roots or the use of a large allograft strut such as a femoral shaft allograft with adjacent fixation to help achieve fusion to the host vertebrae above and below. The disadvantage of this construct is the lack of flexibility in rotation and flexion, the need for healing at two allograft-host junctions, and the risk of migration of the rigid bone graft.

Dental Reconstruction

Dental loss, in addition to the cosmetic disadvantage, is associated with loss of mandibular and maxillary bone as well as dietary limitations. In certain patients, dental implants have been developed. Professor Per-Ingvar Brånemark and coworkers played a major role in the development of such implants. Their techniques emphasized osseointegration of the implant and a two-stage procedure to secondarily load the implant after a period of unloading. Using the standard technique, the base of the implant is inserted into the mandibular or maxillary bone in the appropriate position. After ingrowth of host bone based on radiographs, the overlying gingival tissue is opened and the superstructure is attached.

The complexities and pitfalls of dental implants are dominated by issues of fixation and bone loss. Over time, the supporting bone can be eroded. This can be due to biological factors such as smoking, osteopenia, infection, particulate debris, and/or implant micromotion. Efforts to restore this bone have centered on bone graft techniques and recent application of distraction osteogenesis. For large defects, free tissue transfer and structural bone grafts can be used. These are associated with donor site morbidity, complication rates related to microvascular repair, and inadequate bone incorporation. Alternatively, cancellous bone can be used in some cases but must be contained. Resorbable polylactic acid (PLA) mesh has been utilized as a method to contain the bone graft. Further, Marx et al. have combined bone grafting with dental implants which act as a so-called “tent graft”. The bone graft is placed between the implants as they maintain the height of the construct.

Ferretti et al. have advocated the use of bone morphogenic proteins (BMP's) to reconstruct segmental defects of the mandible. They combined the BMP's with human demineralized bone matrix and combined this with the structural support of a titanium mesh to span the defect. They obtained histologic bone formation in only 2 of 6 patients.

Thus, a need exists for a device that provides structural support, bone ingrowth, and durability, and that is usable for the restoration of bone loss adjacent to a joint, intervertebral disc, or in the oral cavity.

### BRIEF SUMMARY OF THE INVENTION

The present invention provides an implantable device comprising a prosthesis and a flexible member attached to the prosthesis by means of one or more attachment members, where the flexible member is arranged around the prosthesis to form a cavitary space. The cavitary space is then filled with a variety of osteoconductive and osteoinductive materials. The present invention facilitates the restoration of bone loss, including bone loss adjacent to a joint, intervertebral disc, or in the oral cavity, by providing structural support, bone ingrowth, and durability. Also provided are methods of reconstructing skeletal defects with such devices.

In one aspect of the present invention, an implantable device for use in reconstructing osseous skeletal defects is provided, where the device comprises a prosthesis having an attachment member, and a flexible member attached to the prosthesis by means of the attachment member, where a cavitary space is formed between the flexible member and the prosthesis.

In another aspect, a different implantable device for use in reconstructing osseous skeletal defects is provided. This device comprises a prosthesis having an attachment member, and a flexible member having a prosthetic margin, where the prosthetic margin is adapted to fit the attachment member of the prosthesis, and further where attachment of the flexible member to the prosthesis forms a cavitary space between the flexible member and the prosthesis.

In a further aspect of the invention, a method for reconstructing osseous skeletal defects is provided, the method comprising providing a prosthesis having an attachment member and adapted to be affixed into host bone, attaching a flexible member to the prosthesis by means of the attachment member, thereby forming a cavitary space between the flexible member and the prosthesis, and filling the cavitary space with bone graft materials.

In still another aspect of the invention, a different method for reconstructing osseous skeletal defects is provided, this method comprising providing a prosthesis having an attachment member and adapted to be affixed into host bone, providing a flexible member having a prosthetic margin adapted to fit the attachment member, attaching the flexible member to the prosthesis by fitting the prosthetic margin onto the attachment member, thereby forming a cavitary space between the flexible member and the prosthesis, and filling the cavitary space with bone graft materials.

Additional advantages and features of the present invention will be apparent from the following drawings, detailed description and examples which illustrate preferred embodiments of the invention.

### BRIEF DESCRIPTION OF THE DRAWINGS

FIGS. 1A, 1B, and 1C illustrate cross-sectional, top, and perspective views of an embodiment of a flexible member of the present invention.

FIGS. 2A, 2B, and 2C depict side, anteroposterior, and perspective views of an embodiment of a total hip arthroplasty femoral component implant of the present invention.

FIGS. 3A, 3B, 3C, and 3D show side, anteroposterior, and front and rear perspective views of the implant of FIG. 2 embedded into a patient's femur, with a flexible member attached to the implant.

FIGS. 4A, 4B, 4C, and 4D illustrate an acetabular reconstruction using an implantable device of the present invention, where FIG. 4A illustrates an acetabular implant, FIG. 4B illustrates a bone defect in a patient's hip, and FIGS. 4C and 4D depict the acetabular implant positioned into a patient's hip.

FIGS. 5A, 5B, 5C, and 5D depict top, side, front, and perspective views of an embodiment of a femoral component implant with a long stem for distal femoral reconstruction.

FIGS. 6A, 6B, and 6C show front, side, and perspective views of the implant of FIG. 5, with a flexible member attached to the implant.

FIGS. 7A, 7B, and 7C illustrate side, anteroposterior, and perspective views of an embodiment of a tibial component implant of the present invention.

FIGS. 8A, 8B, and 8C depict anteroposterior, side, and perspective views of the implant of FIG. 7 embedded into a patient's tibia, with a flexible member attached to the implant.

FIGS. 9A, 9B, and 9C show side, top, and perspective views of an embodiment of a spinal implant of the present invention.

FIGS. 10A and 10B illustrate side and perspective views of the spinal implant of FIG. 9, with a flexible member attached to the implant.

FIG. 11 is a schematic view of an embodiment of a dental implant of the present invention.

FIGS. 12A and 12B are superior perspective and inferior perspective views of the dental implant of FIG. 11, prior to attachment of a superior segment of the implant (crown).

FIGS. 13A, 13B, 13C, and 13D illustrate side, top, superior perspective and inferior perspective views of the dental implant of FIG. 11, after attachment of the superior segment of the implant (crown).

FIGS. 14A, 14B, and 14C depict front perspective, top, and side views of three dental implants of FIG. 13 embedded into a patient's mandible.

FIGS. 15A, 15B, and 15C depict front perspective, top, and side views of the dental implants of FIG. 14, with flexible members attached to the implants.

FIGS. 16A, 16B, 16C, and 16D illustrate a dental reconstruction using an implantable device of the present invention, where FIG. 16A illustrates an normal human mandible, FIG. 16B illustrates a bone defect in a patient's mandible, and FIGS. 16C and 16D depict the dental implant and flexible member positioned into a patient's mandible.

### DETAILED DESCRIPTION OF THE INVENTION

Reference will now be made in detail to the presently preferred embodiments of the invention, which, together with the following examples, serve to explain the principles of the invention. These embodiments are described in sufficient detail to enable those skilled in the art to practice the invention, and it is to be understood that other embodiments may be utilized, and that structural, chemical, and biological changes may be made without departing from the spirit and scope of the present invention.

The present invention relates to an implantable device comprising a prosthesis and a flexible member attached to the prosthesis by means of one or more attachment members, where the flexible member is arranged around the prosthesis to form a cavitary space. The cavitary space is then filled with a variety of osteoconductive and osteoinductive materials. The present invention facilitates the restoration of bone loss, including bone loss adjacent to a joint, intervertebral disc, or in the oral cavity, by providing structural support, bone ingrowth, and durability.

The prosthesis may be any skeletal prosthesis such as a joint arthroplasty implant, an artificial disc implant, or a dental implant, modified by the addition of attachment members to facilitate attachment of the flexible member thereto. The present invention can be utilized with any type of orthopaedic implant as long as the desired position of the implant facilitates attachment of the attachment member. Orthopaedic protheses are manufactured by a large number of corporations, including Zimmer, Warsaw, Ind.; Biomet, Warsaw, Ind.; and Smith and Nephew, Memphis, Tenn. Most mechanical prostheses in current use in total joint replacements are manufactured from alloys such as cobalt-chromium, or made of titanium.

If a standard orthopaedic prosthesis is used in the methods of the present invention, it is modified to attach an attachment member to the prosthesis. One embodiment of an attachment member includes the use of metal rails welded to the prosthesis to which a flexible member can be interlocked at one end and then banded to the residual host bone at the other end. Other methods of attachment can be with the use of screws, pins, bands, and/or sutures to interlock the flexible member to the prosthesis.

The prosthesis can be fixed into the patient's native skeleton, or for a spinal prosthesis can be linked in a chain-like fashion to an adjacent artificial disc. In the case of a joint prosthesis, the articulating surface is stabilized at a given distance from the residual bone to reconstruct the joint at the appropriate level. The prosthesis can be embedded in the host bone using any mechanical fixation necessary. Modes of fixation can be with the use of methylmethacrylate bone cement or by ingrowth of bone into the prosthesis.

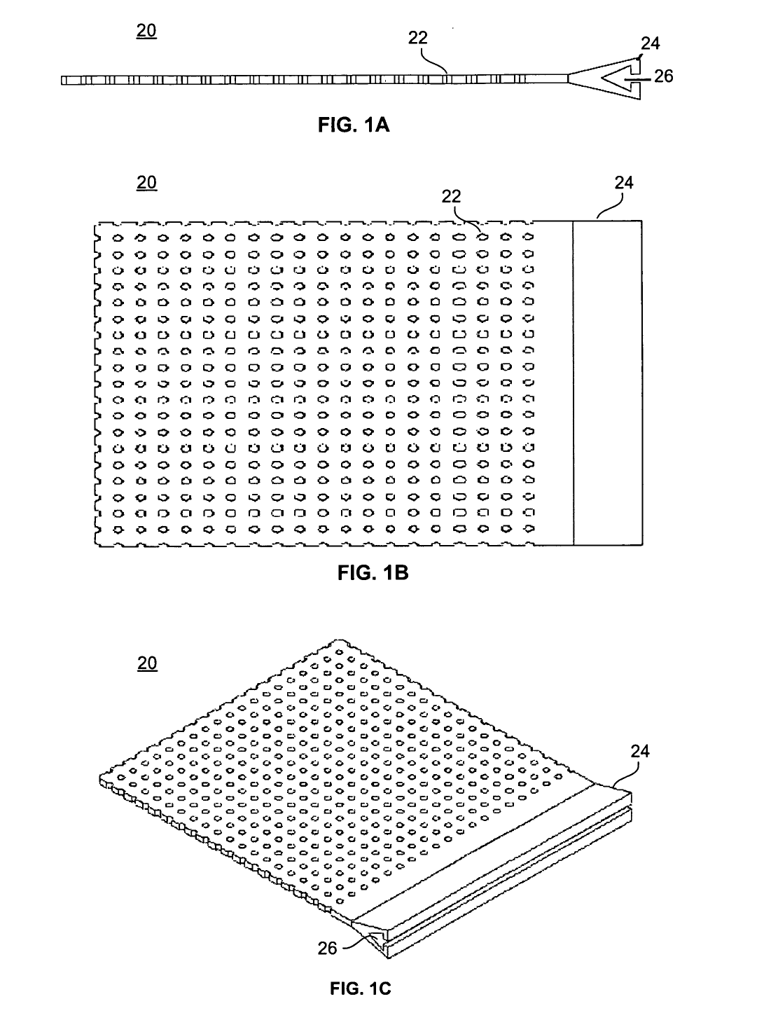

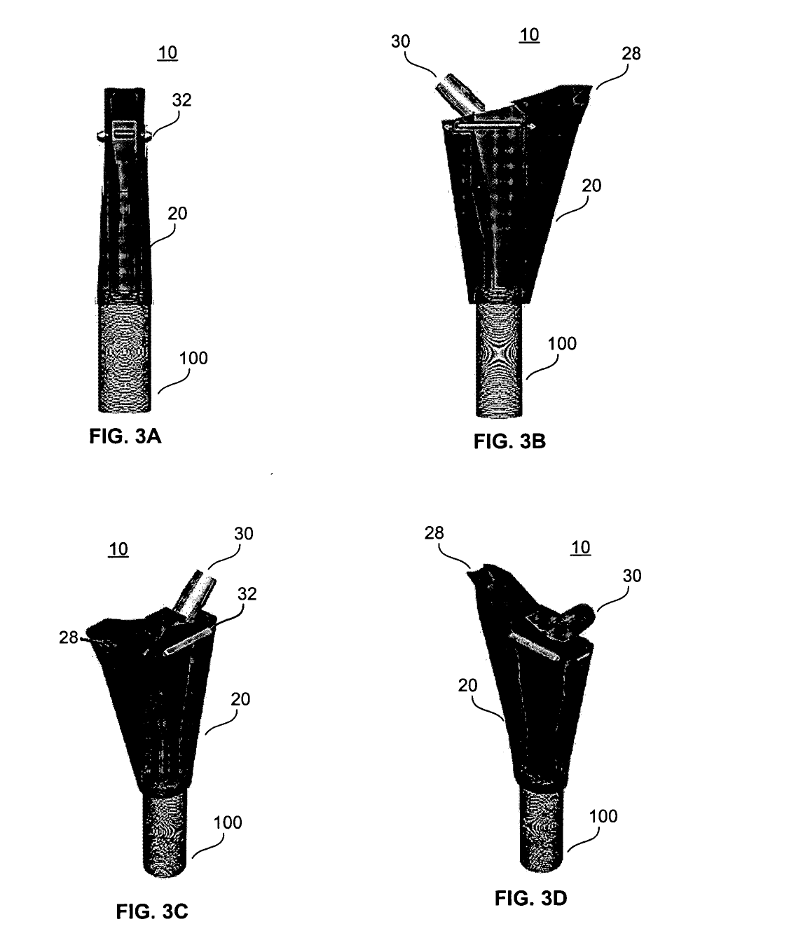

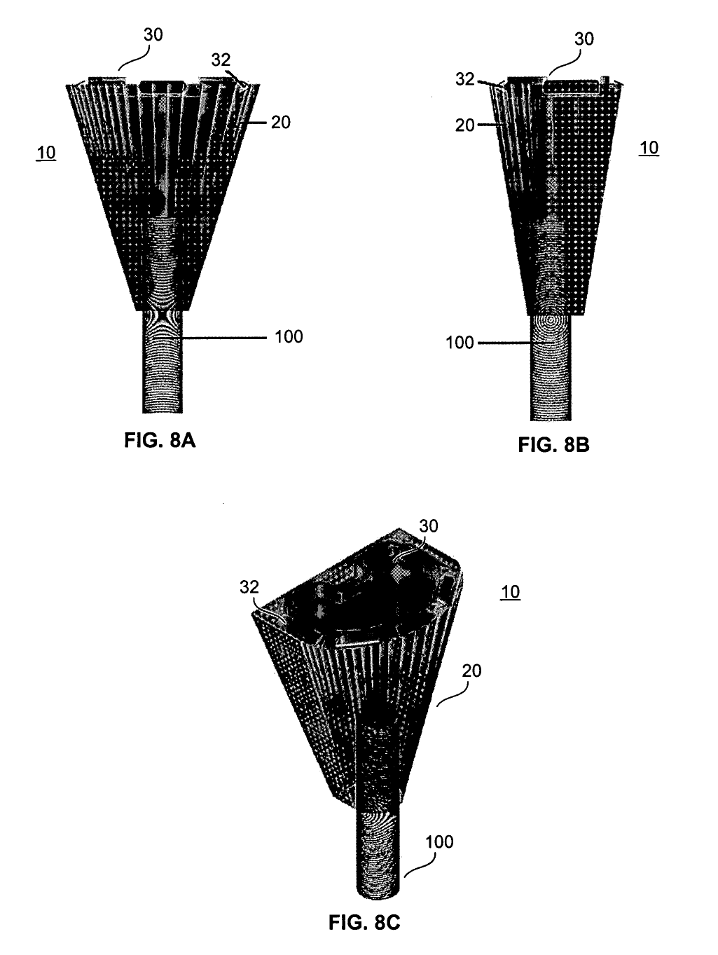

Referring now to the Figures, the flexible member 20 may have any suitable generic shape, such as that of a oblong sheet or mesh (as can be seen in FIGS. 1B and 1C), or may be particularly shaped to fit a particular prosthesis (as shown in FIGS. 3 and 8). Regardless of the shape, the flexible member 20 is perforated to allow ingress of blood vessels during the maturation process of the reconstituted bone. The perforations (or holes) 22 are between about 100 to about 2000 microns in diameter and spaced at a distance of about 1000 to about 10,000 microns depending on the specific application. Although the perforations 22 are shown in FIG. 1 as regularly spaced and of the same size, they may be randomly placed, and may be of different sizes. In other embodiments, the flexible member 20 may be a fibrous network or a wire mesh, instead of as a perforated sheet.

The flexible member is at least flexible enough to permit a surgeon to make appropriate adjustments during implantation, but need not be substantially flexible after implantation, and may, for example, be treated after shaping and/or implantation to hold to a particular shape (such as, for example, by UV curing). The flexible member must be of sufficient tensile strength to maintain its attachments to the prosthesis and to the host bone, particular when filled with the bone graft material.

As desired for a particular application, the flexible member may be bioresorbable or non-resorbable, and may be formed from metal, a biomaterial such as demineralized bone matrix, or a polymer. In a preferred embodiment, the flexible member is formed of a resorbable polymer such as polylactic acid (PLA), polyglycolic acid (PGA), collagen, hyaluronate, demineralized bone matrix, or any one of a number of other flexible or semi-rigid materials.

For many applications, a resorbable flexible member is preferred. During the maturation process of the contained bone graft material, a resorbable flexible member will be nearly completely metabolized, with the potential to reconstitute an outer periosteal layer for the new bone and to allow further vascular perforation of the bone graft. In other applications, a non-resorbable flexible member such as metal mesh is preferred. These circumstances include cases in which mechanical loading of the flexible member is required. For example, in the case of an acetabular reconstruction, a flexible member composed of wire mesh rather than a resorbable polymer can be used to contain the bone graft under high compressive pressure until it matures around a porous ingrowth acetabular (hip socket) component.

The flexible member is attached to the prosthesis by means of one or more attachment members, non-limiting examples of which include rails, runners, and suture holes. In a preferred embodiment, the prosthesis has triangular rails affixed in key locations, and the flexible member has a prosthetic margin designed to match or mate the triangular rails. The shape of the rails, and the corresponding shape of the prosthetic margin, is not limited to a triangular cross-section, but may be any suitable geometric shape allowing for a secure interlock.

Referring again to FIG. 1, the prosthetic margin 24 can be formed by a thickening of the flexible member 20 with a receptacle 26 for the triangular rails of the prosthesis. The receptacle 26 has an identical cross-sectional geometry to the rails with slightly larger dimensions to allow interlocking of the sheet to the rail. Alternatively, the flexible member 20 can be fixed to the prosthesis (not shown in this Figure) with some other form of fixation such as an adhesive, suture or clip.

In a preferred method of use, the prosthesis is first fixed to the patient's host bone by standard surgical means. After initial stabilization of the prosthesis to the host is achieved, the flexible member is wrapped around the prosthesis by attaching it to one or more attachment members on the prosthesis. The subsequent cavitary space located between the outer surface of the prosthesis and the inner surface of the flexible member is then filled with any of a variety of osteoconductive and osteoinductive materials. Non-limiting examples of such materials include autologous bone graft (from the patient), cancellous bone allograft (from a cadaver donor), and bone graft substitutes such as calcium sulfate, calcium carbonate, calcium phosphate, hydroxyapatite, demineralized bone, and/or bone morphogenic protein (BMP). Calcium sulfate is available from Wright Medical (Arlington, Tenn.), hydroxyapatite is available from Interpore-Cross (Irvine, Calif.), and demineralized bone and bone morphogenic protein are available from Stryker (Kalamazoo, Mich.). Calcium carbonate and calcium phosphate are available from standard medical suppliers.

The flexible member is then attached to the host bone using resorbable or non-resorbable clips, pins, screws, cables, or bands thus containing the bone graft and allowing it to mature around the prosthesis. In a particularly preferred method, a resorbable bone screw with a thread matching the specific sheet pore size (for pore sizes greater than 1000 microns), is used to attach the flexible member to the host bone.

The outer surface of the metal prosthesis is composed of an ingrowth surface which can be comprised of a porous metal, ceramic, or other surface. This allows stable fixation to the host residual bone. The contained bone graft matures in a pattern dictated by the contour of the flexible member, healing to the residual host bone and optimally achieving ingrowth or ongrowth onto the prosthesis. Thus it reconstructs the osseous defect from the level of the residual host bone to the level of the prosthesis.

In essence the flexible member acts as a periosteum dictating the shape and size of the reformed bone adjacent to the articulating surface, tooth, or artificial disc implant. As this bone graft is loaded around the prosthesis, it is exposed to stresses that further drive it to remodel according to Wolff's Law. Wolff's Law refers to the tendency of bone to respond with increased density and strength when exposed to a compressive load. The flexible member affords additional stability around the bone graft by containing it and providing a column of support for the articulating portion of the prosthesis.

Application of the teachings of the present invention to a specific problem or environment is within the capabilities of one having ordinary skill in the art in light of the teachings contained herein. Examples of the products and processes of the present invention appear in the following examples.

### EXAMPLE 1

Femoral Resection

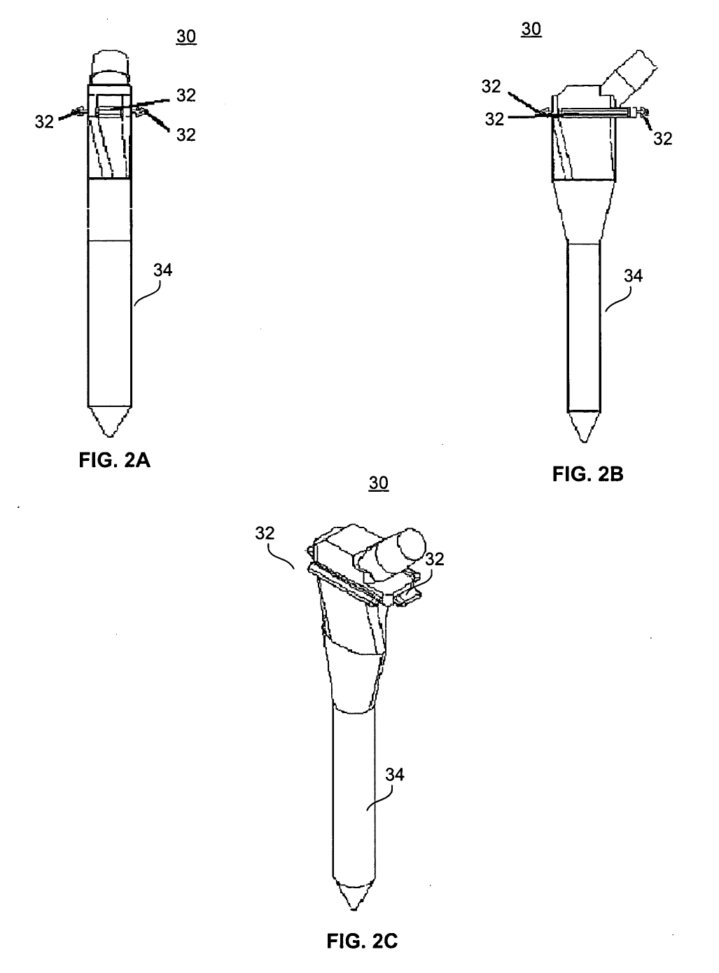

With reference to FIGS. 2 and 3, the present invention is utilized in resection of a proximal femoral osteosarcoma in a 15 year old male. FIGS. 2A through 2C illustrate a femoral prosthesis 30 of the present invention, and FIGS. 3A through 3D illustrate an implantable device 10 comprising the femoral prosthesis 30 surrounded by a flexible member 20. The implantable device 10 is used to reconstruct the proximal femur of a patient (not shown) in a five-step process. This process is adaptable for use, as will be evident to those of skill in the art, within any of the large joints including the hip, knee, shoulder, elbow, and ankle.

First, a prosthesis 30 is selected for use, with consideration given to the appropriate height of the stem 34 in order to achieve adequate leg length and soft tissue tension in the patient. The prosthesis 30 is provided with one or more attachment members 32, which are placed circumreferentially around the proximal end of the prosthesis. The stem 34 of the prosthesis 30 is implanted into the host bone 100 by typical surgical means, such as press-fitting, and diaphyseal fixation.

Second, a flexible member 20 is provided for use with the prosthesis 30, and this flexible member preferably is constructed to match the planned three-dimensional shape and structure of the reconstructed proximal femur, i.e., a greater and lesser trochanter. The flexible member 20 is affixed to the attachment members 32, such as by mechanically bonding, i.e., interlocking, the prosthetic margin 26 (as shown in FIG. 1) onto the attachment members 32. Then, the flexible member 20 is wrapped or tubularized around the prosthesis 30 to form a cavitary space between the flexible member 20 and the prosthesis 30. Any excess flexible member 20 may be trimmed or cut.

Third, tendons (not shown) such as the hip abductors and patellar tendon are attached to the implantable device 10. Either the tendon as a soft tissue structure, or with its bony attachment, is attached, such as with sutures, to the flexible member 20 or to the prosthesis 30. If the tendon is attached to the prosthesis 30, it is first passed through aperture 28 in the flexible member 20. The attachment of the tendons facilitates the formation of Sharpey's fibers into the reconstituted proximal femur.

Fourth, the cavitary space formed between the flexible member 20 and the prosthesis 30 is filled with osteoconductive or osteoinductive material. Non-limiting examples of suitable filler material include autologous bone graft, cancellous bone allograft, and bone graft substitutes such as calcium sulfate, calcium carbonate, calcium phosphate, hydroxyapatite, demineralized bone, and/or bone morphogenic proteins.

Fifth and lastly, the free margin of the flexible member 20 is attached to the host femur 100. Fixation is achieved by suitable surgical means known to those of skill in the art, including drill holes and sutures, a circumferential band, small resorbable screws, or any method that will maintain the containment of the bone graft within the flexible member.

### EXAMPLE 2

Acetabular Reconstruction

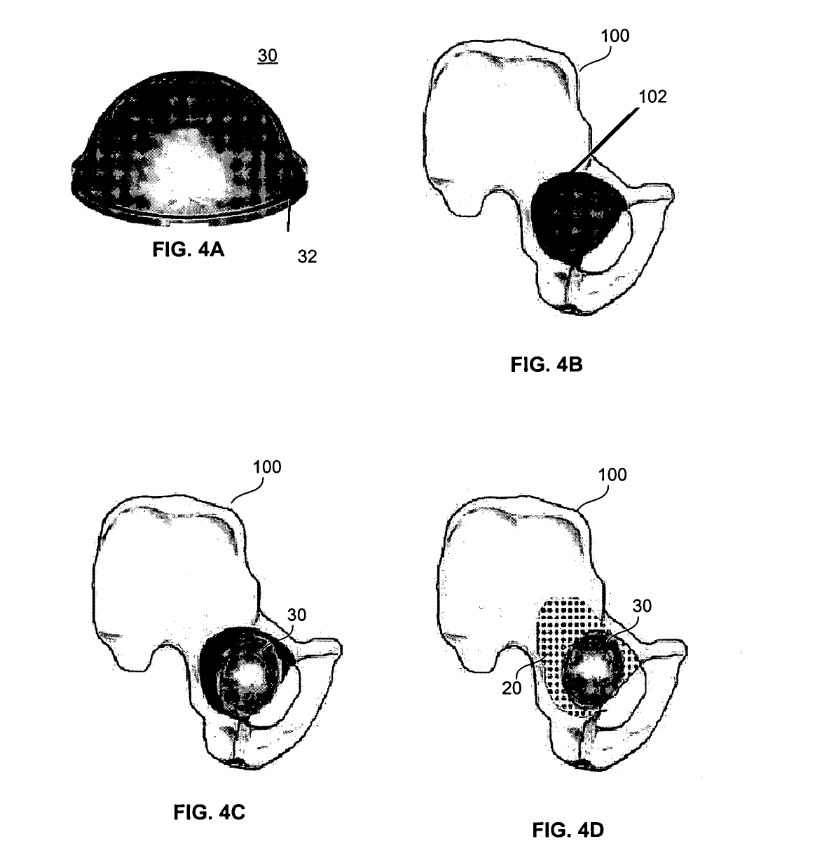

With reference to FIG. 4, the present invention is used to treat a large superior defect 102 of the acetabulum 100 in the case of hip dysplasia or in the revision setting. FIG. 4A shows a porous surface uncemented cup prosthesis 30. FIG. 4B depicts a patient's acetabulum 100 with a large superior dome defect 102. As shown in FIG. 4C, the cup 30 is fixed to the residual acetabulum using acetabular screws, or a combination of modular cup attachments for screw fixation to the ilium, ischium, and pubis.

The residual bone loss is reconstituted by attachment of the flexible member 20 to the margins of the cup 30 with attachment members 32, as shown in FIG. 4D, and by filling the resultant cavitary space with bone graft material, as described in Example 1. This bone graft has the potential to mature into a vascularized bed that can grow into the porous surface of the prosthesis and also facilitate any future acetabular revision surgeries. The free margins of the flexible member 20 are then attached to the ilium using bioadsorbable or metal screws.

### EXAMPLE 3

Total Knee Arthroplasty

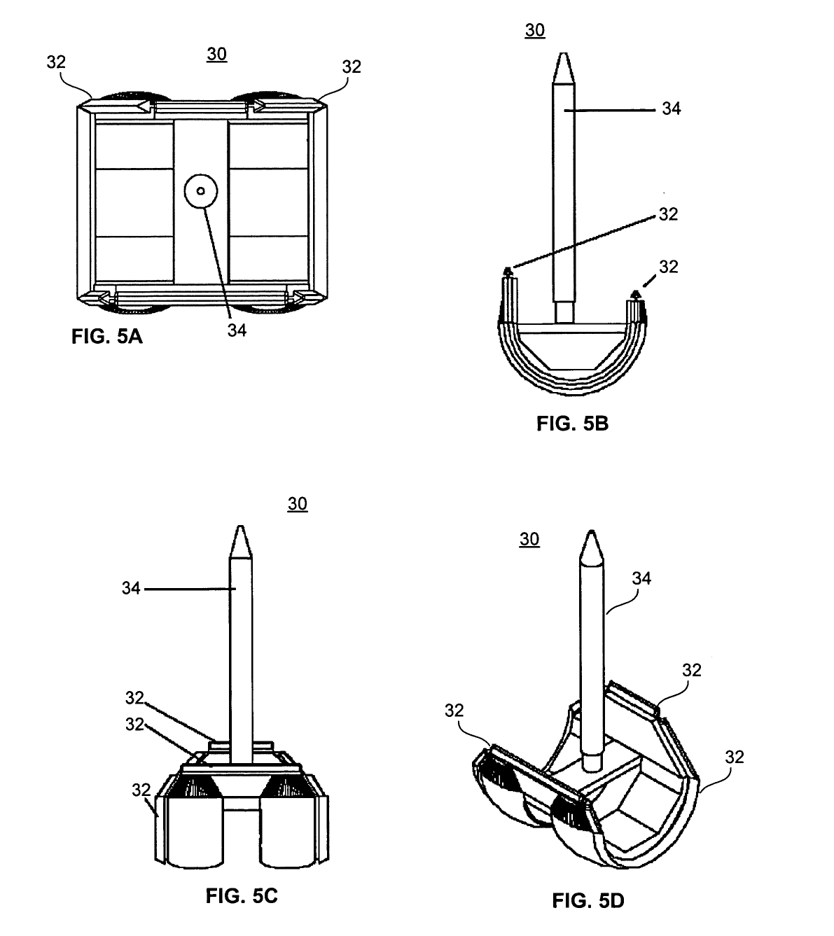

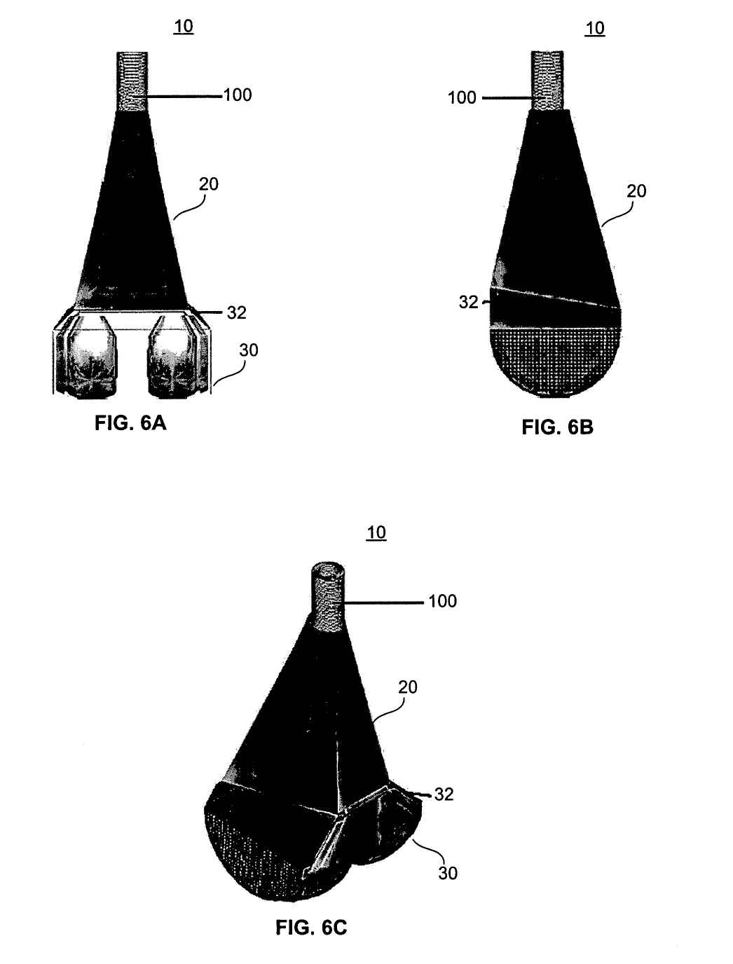

With reference to FIGS. 5 and 6, a total knee arthroplasty with a comminuted supracondylar fracture with major bone loss is treated with a long press-fit intramedullary revision femoral component embedded in the residual femoral diaphysis. FIGS. 5A through 5D illustrate a femoral prosthesis 30 of the present invention, and FIGS. 6A through 6C illustrate an implantable device 10 comprising the femoral prosthesis 30 surrounded by a flexible member 20. The implantable device 10 is used to reconstruct the distal femur of a patient (not shown) in a multi-step process.

The process described in Example 1 is adapted for use on the distal femur, wherein first a prosthesis 30 is selected for use, with consideration given to the appropriate height and circumference of the stem 34. The prosthesis 30 is provided with one or more attachment members 32, to which the flexible member 20 is attached. The proximal margins of the flexible member 20 are fixed to the outer surface of the femoral diaphysis 100 enclosing a space which is filled with cancellous bone allograft and bone morphogenic protein. The cancellous bone matures over time and achieves bone fixation to the prosthesis ingrowth surface, thereby avoiding the use of an allograft and restoring native bone to the patient's distal femur.

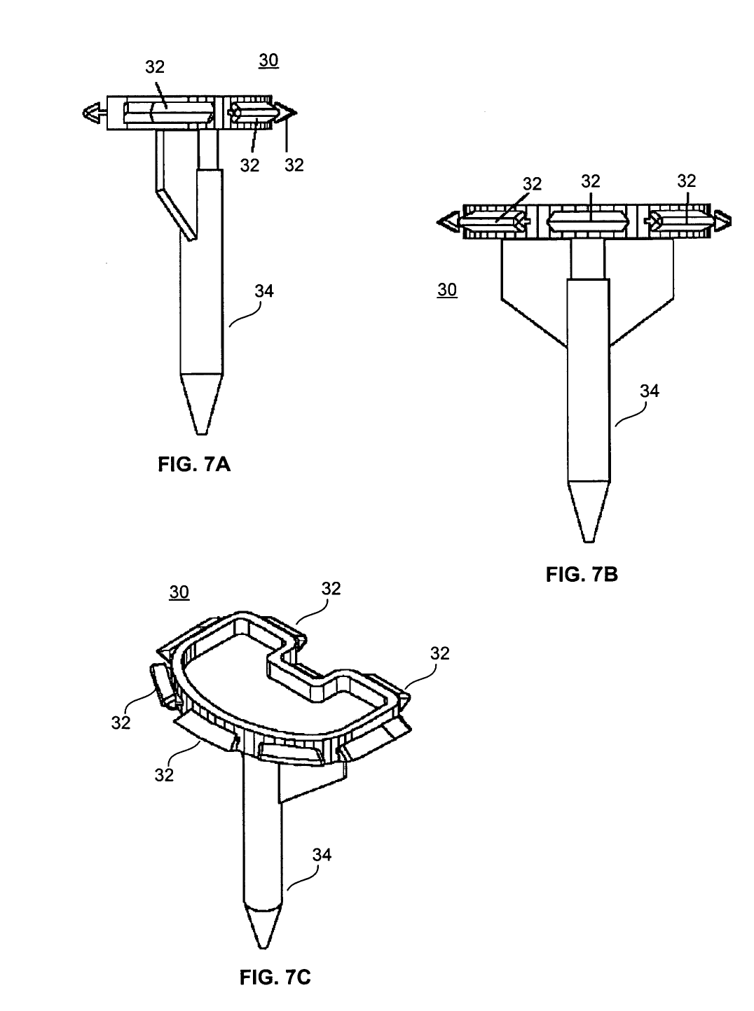

FIGS. 7 and 8 illustrate the tibial component of the total knee arthroplasty. FIGS. 7A through 7C illustrate the tibial prosthesis 30, and FIGS. 8A through 8C illustrate an implantable device 10 comprising the tibial prosthesis 30 surrounded by a flexible member 20. The implantable device 10 is used to reconstruct the tibia of a patient (not shown) in a multi-step process as described above and in Example 1.

### EXAMPLE 4

Spinal Reconstruction

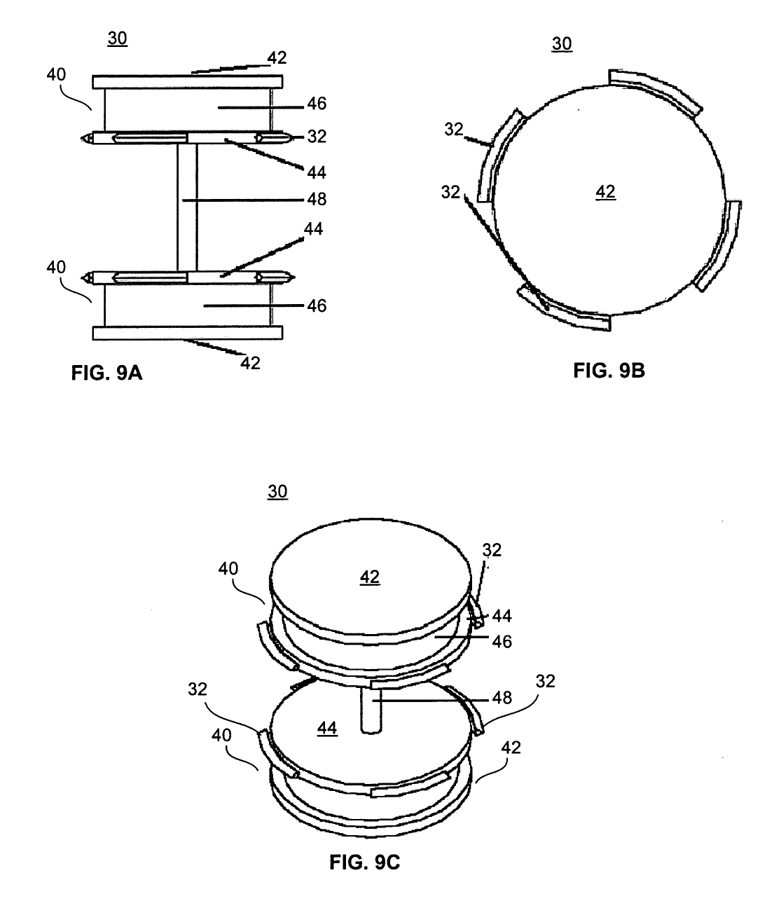

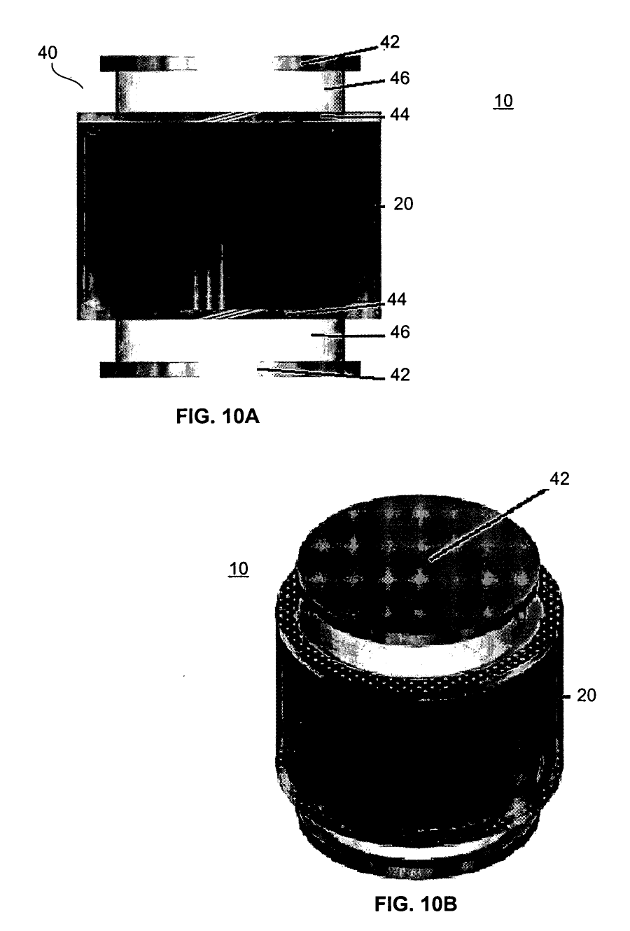

With reference to FIGS. 9 and 10, the present invention is utilized to treat bone loss in the spine. A young patient with a tumor of L4 vertebral body is treated with a composite implant with an artificial disc attached to the distal endplate of L3 and the proximal endplate of L5. FIGS. 9A through 9C illustrate a spinal prosthesis 30 of the present invention, and FIGS. 10A and 10B illustrate an implantable device 10 comprising the spinal prosthesis 30 surrounded by a flexible member 20.

The spinal prosthesis 30 comprises two interconnected artificial discs 40, each composed of two endplates 42, 44 and a central bearing 46 corresponding in size to an intervertebral disc. The artificial discs 40 are linked with a central support rod 48. The endplates 42, 44 are composed of metal implant material, with the superior surface of exterior endplates 42 being composed of an ongrowth metal surface. The central bearing 46 is composed of any desired material that mimics the cushioning and support of a native disc and is suitable for implantation, such as polyethylene or other polymer materials.

The margins of the internal endplates 44 contain attachment members 32 for attachment of the flexible member 20 in a circumferential fashion. The cavitary space formed between the flexible member 20 and the prosthesis 30 is filled with bone graft and the flexible member 20 is attached onto itself, thereby enclosing the bone graft. This facilitates restoration of the vertebral body and eventual unloading of the support column, preventing fatigue failure. The contralateral endplates 42 are attached to the patient's remaining vertebrae (not shown) using a standard technique depending on the implant design.

### EXAMPLE 5

Dental Implants

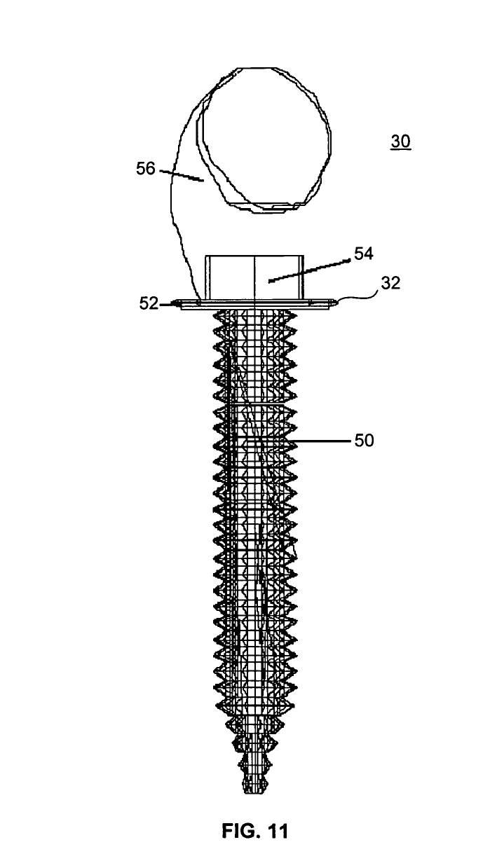

With reference to FIGS. 11 through 16, the present invention is used with the gingival margin of the implantable device corresponding to the articular surface of the orthopedic implantable device. In an embodiment applied to the oral cavity, the invention is applied to a 60 year old smoker with an oral carcinoma necessitating partial glossectomy and removal of the posterior mandibular molar and premolars along with 50 percent of the height of the mandibular bone. The use of a standard implant, or other standard techniques such as distraction osteogenesis would be difficult in such a case due to the large dimension of the bone loss in the sagittal plane.

FIGS. 11 through 13 illustrate a dental prosthesis 30 of the present invention having a central stem 50 composed of metal, and capable of being affixed to native bone (not shown) using a screw-in or press-fit technique. A base plate 52 is fixed to and above the central stem 50, and is composed of metal. Attachment members 32 are affixed to the periphery of the base, and may either be a single member running along the entire periphery of the base plate 52, or may comprise multiple members arranged about the periphery.

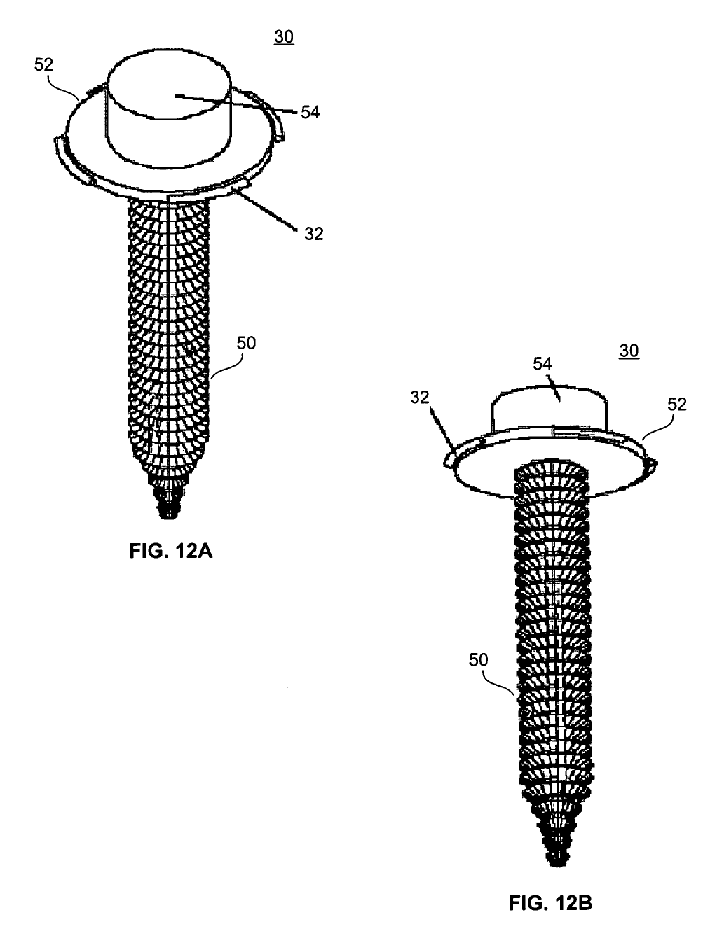

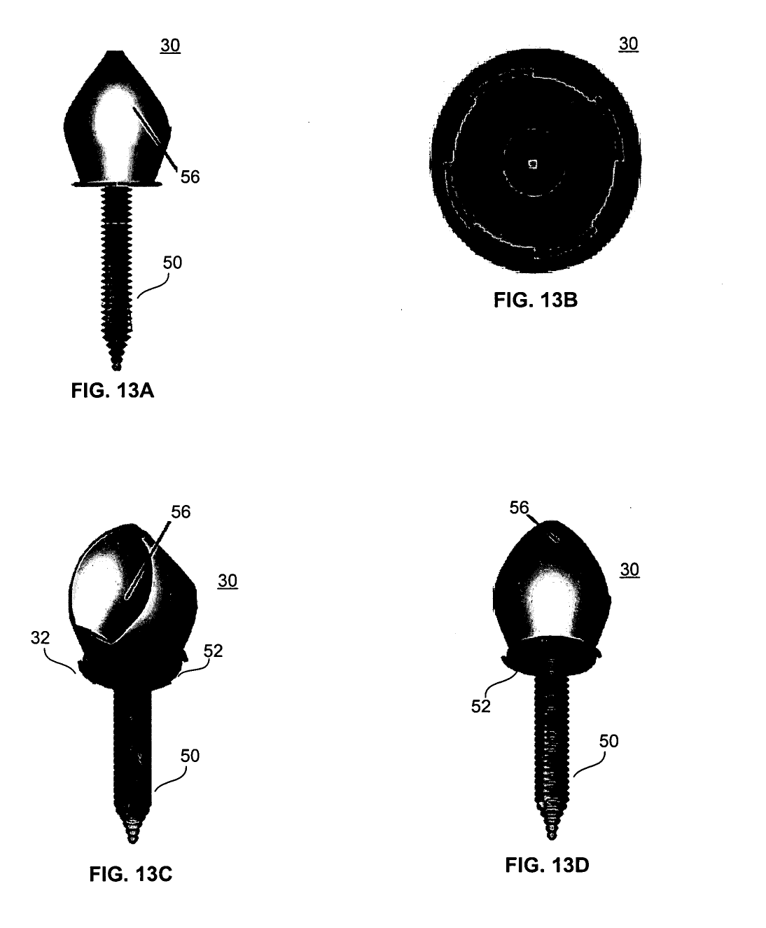

A support post 54 located on the base plate 52 permits the fixation of a superior segment 56 to the rest of the prosthesis. The superior segment 56 can be a standard dental crown. FIGS. 11 and 13 depict the prosthesis with the superior segment 56 attached, and FIG. 12 depicts the prosthesis without the superior segment 56 attached.

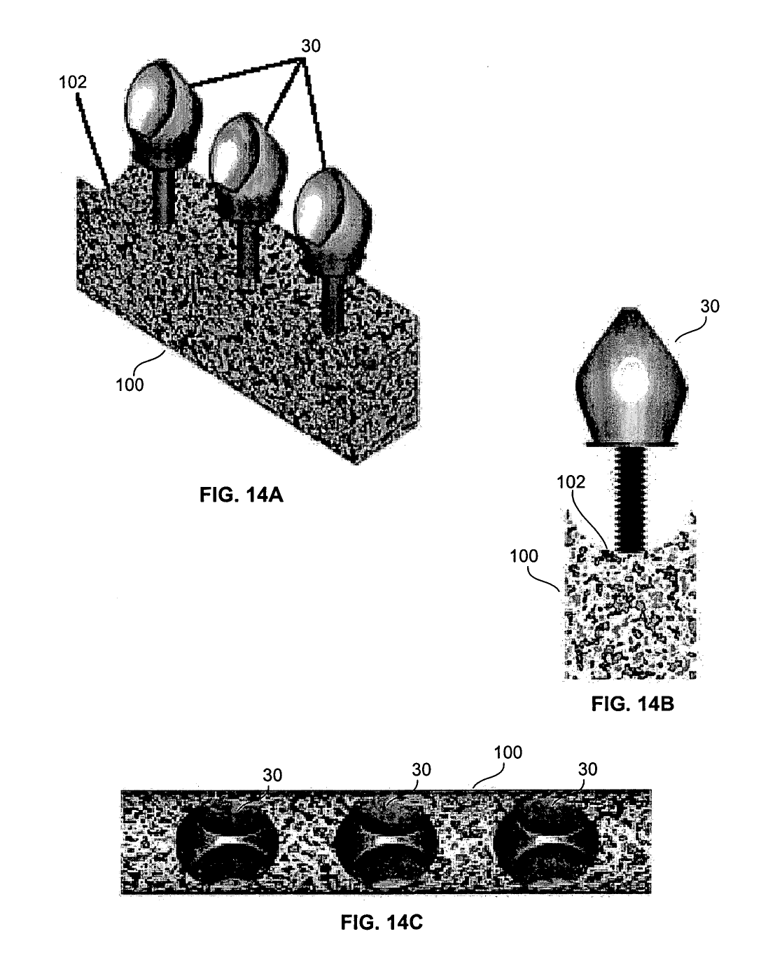

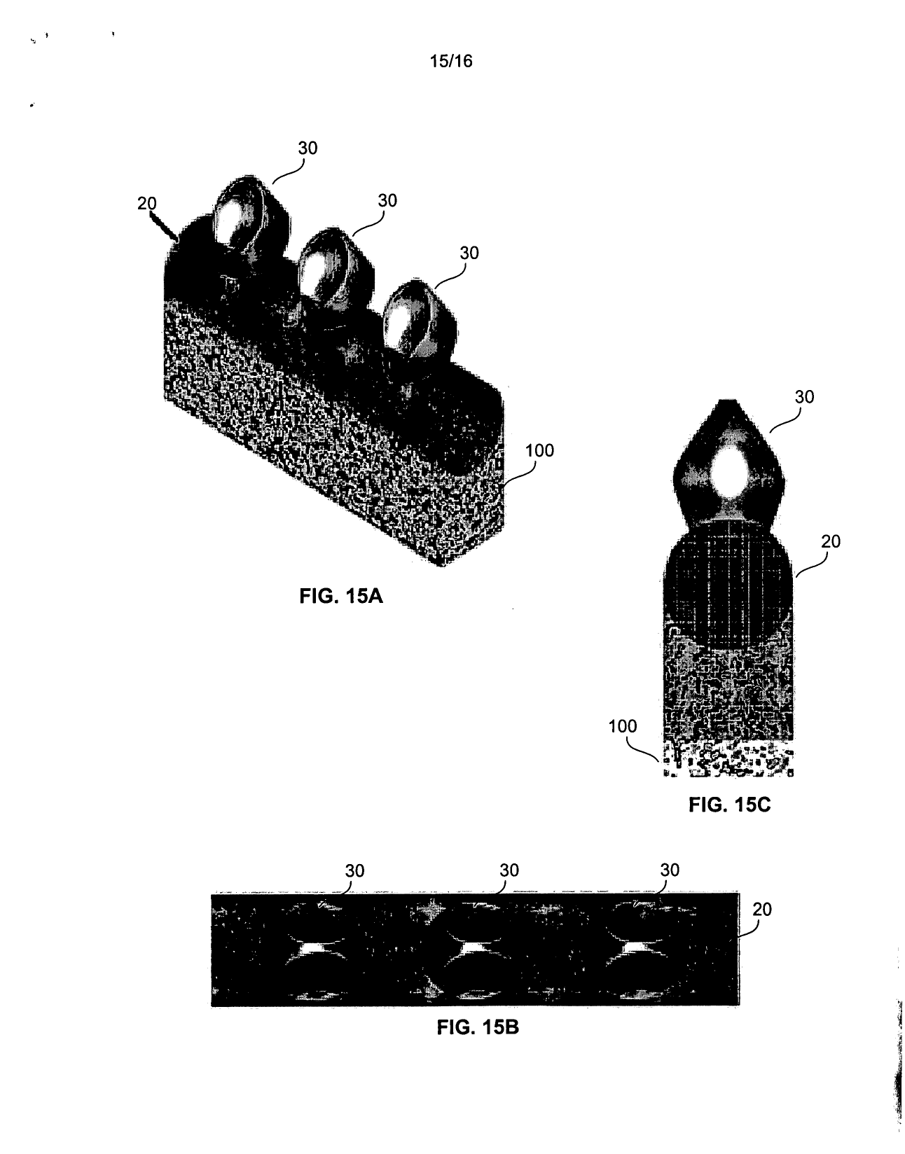

FIG. 14 illustrates schematically the fixation of three dental prostheses 30 into a concave cavitary mandibular defect 102 of a patient's mandible or maxilla 100. In FIG. 15, a flexible member 20 is attached to the dental prostheses 30, with a single flexible member 20 spanning the entire defect. The cavitary space formed between the flexible member 20 and the prostheses 30 is filled with bone graft material, and the free ends of the flexible member 20 are then affixed to the patient's mandible or maxilla 100 using screws, staples, sutures, or alternative fixation elements (not shown). Once the construct is fully mature based on radiographs, the superior members 56 are attached to the implants, thereby reconstituting the dental architecture and restoring the mandibular bone.

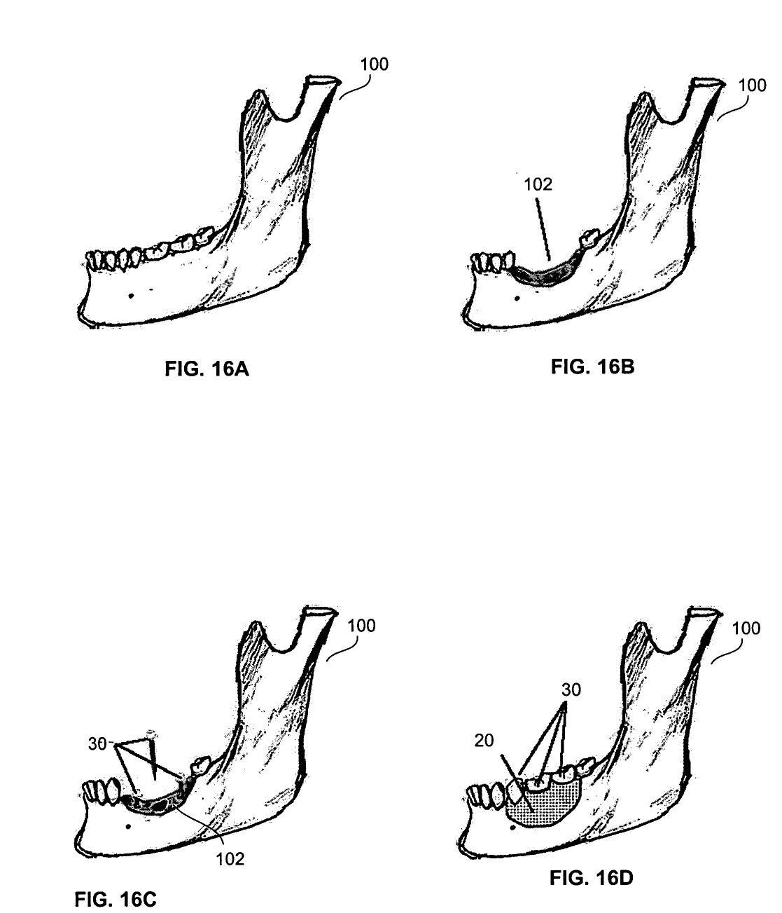

With reference to FIG. 16, the implantable device of the present invention is used to treat a large osseous defect 102 of the mandible 100. FIG. 16A shows a normal human mandible 100. FIG. 16B depicts a patient's mandible 100 with dental excision and a large osseous defect 102. As shown in FIG. 16C, dental prostheses 30 are fixed to the residual bone 100, such as by pre-drilling the recipient site and press-fitting the prosthesis into the pre-drilled site. In FIG. 16D, one or more flexible members 20 are then attached to the prostheses 30, filled with bone graft, and fastened to the mandible 100 using suitable fixation elements (not shown).

The foregoing disclosure of the preferred embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

1. An implantable device for use in reconstructing osseous skeletal defects comprising:
a prosthesis having an attachment member; and
a flexible member attached to said prosthesis by means of the attachment member, wherein a cavitary space is formed between said flexible member and said prosthesis.

2. The implantable device of claim 1, wherein said prosthesis is selected from the group consisting of a spinal prosthesis, a joint arthroplasty prosthesis, and a dental prosthesis.

3. The implantable device of claim 2, wherein the joint arthroplasty prosthesis is selected from the group consisting of a femoral stem prosthesis, an acetabular cup prosthesis, a distal femur prosthesis, and a tibial prosthesis.

4. The implantable device of claim 1, wherein the attachment member is a triangular rail.

5. The implantable device of claim 1, wherein the attachment member is a suture hole.

6. The implantable device of claim 1, further comprising bone graft materials located within the cavitary space.

7. The implantable device of claim 6, wherein the bone graft materials comprise osteoconductive materials, osteoinductive materials, or both osteoconductive materials and osteoinductive materials.

8. The implantable device of claim 6, wherein the bone graft materials are selected from the group consisting of cancellous bone allograft chips, calcium sulfate, calcium carbonate, calcium phosphate, hydroxyapatite, and demineralized bone matrix.

9. An implantable device for use in reconstructing osseous skeletal defects comprising:
a prosthesis having an attachment member; and
a flexible member having a prosthetic margin, wherein the prosthetic margin is adapted to fit the attachment member of said prosthesis, and further wherein attachment of the flexible member to the prosthesis forms a cavitary space between said flexible member and said prosthesis.

10. The implantable device of claim 9, wherein said flexible member comprises metal, demineralized bone matrix, or a polymer.

11. The implantable device of claim 9, wherein said flexible member comprises a resorbable polymer.

12. The implantable device of claim 11, wherein the resorbable polymer is selected from the group consisting of polylactic acid, polyglycolic acid, collagen, and hyaluronate.

13. The implantable device of claim 9, wherein said flexible member is perforated.

14. The implantable device of claim 9, wherein said flexible member is a fibrous network.

15. The implantable device of claim 9, wherein said flexible member is a wire mesh.

16. The implantable device of claim 9, wherein the attachment member is a triangular rail, and wherein said prosthetic margin has a receptacle adapted to interlock with the triangular rail.

17. A method for reconstructing osseous skeletal defects comprising:
providing a prosthesis having an attachment member and adapted to be affixed into host bone;
attaching a flexible member to the prosthesis by means of the attachment member, thereby forming a cavitary space between the flexible member and the prosthesis; and
filling the cavitary space with bone graft materials.

18. A method for reconstructing osseous skeletal defects comprising:
providing a prosthesis having an attachment member and adapted to be affixed into host bone;
providing a flexible member having a prosthetic margin adapted to fit the attachment member;
attaching the flexible member to the prosthesis by fitting the prosthetic margin onto the attachment member, thereby forming a cavitary space between the flexible member and the prosthesis; and
filling the cavitary space with bone graft materials.

19. The method of claim 18, wherein said step of providing a prosthesis comprises providing a joint arthroplasty prosthesis, and wherein said step of providing a flexible member comprises providing a flexible member that has a shape of a natural bone.

20. The method of claim 19, wherein the joint arthroplasty prosthesis and its corresponding flexible member are selected from the group consisting of a femoral stem prosthesis and a flexible member having the shape of a natural proximal femur bone, a distal femur prosthesis and a flexible member having the shape of a natural distal femur bone, and a tibial prosthesis and a flexible member having the shape of a natural tibial bone.

21. The method of claim 18, wherein said step of providing a prosthesis comprises providing a prosthesis selected from the group consisting of an acetabular cup prosthesis, a spinal prosthesis, and a dental prosthesis.
